(19) United States
(12) Patent Application Publication
(10) Pub. No.: US 2012/0283334 A1
(43) Pub. Date: Nov. 8, 2012

(54) Treatment of Viral Infections

(75) Inventors: John Brew, London (GB); Robin Mark Bannister, London (GB); Gregory Alan Soloff, London (GB); Wilson Capparros Wanderlay, London (GB); Olga Pleguezuelos Mateo, London (GB)

(73) Assignee: Biocopea, Ltd., London (GB)

(21) Appl. No.: 13/390,040

(22) PCT Filed: Aug. 10, 2010
(86) PCT No.: PCT/GB2010/051317

§ 371 Date: Jul. 24, 2012

(30) Foreign Application Priority Data
Aug. 10, 2009 (GB) 0913914.8
Feb. 4, 2010 (GB) 1001821.6
Jul. 20, 2010 (GB) 1012168.9

(51) Int. Cl. A61K 31/137; A61P 31/16; A61P 31/22

(52) U.S. Cl. 514/653; 514/654

(57) ABSTRACT

The invention provides compositions, medicaments and methods of treatment of viral infections, especially respiratory disorders caused by viral infections. In particular, the invention relates to the treatment of acute viral infections using a range of related 1-phenyl-2-amino ethanol, ethanal, and ethane derivatives.

This 35 U.S.C. §371 application is a national stage filing of PCT/GB2010/051317, filed Aug. 10, 2010 and claims priority pursuant to 35 U.S.C. §119(d) to GB 1012168.9, filed Jul. 20, 2010, GB 1001821.6, filed Feb. 4, 2010, and GB 0913914.8, filed Aug. 10, 2009, each of which is hereby incorporated by reference in its entirety.

The present invention relates to the treatment of viral infections, and especially the treatment of respiratory disorders caused by viral infections. In particular, the invention relates to the treatment of acute viral infections using a range of related 1-phenyl-2-amino ethanol, ethanal and ethane derivatives, and to the use of these compounds in methods of treatment. The invention is particularly concerned with the treatment of respiratory disorders caused by infections with influenza viral strains, including not only existing viruses, but also future, derivative strains of viruses that have mutated from existing viruses, which could give rise to an influenza pandemic.

The defence against disease is critical for the survival of all animals, and the mechanism employed for this purpose is the animal immune system. The immune system is very complex, and involves two main divisions, (i) innate immunity, and (ii) adaptive immunity. The innate immune system includes the cells and mechanisms that defend the host from infection by invading organisms, in a non-specific manner. Leukocytes, which are involved with the innate system, include inter alia phagocytic cells, such as macrophages, neutrophils and dendritic cells. The innate system is fully functional before a pathogen enters the host.

In contrast, the adaptive system is only initiated after the pathogen has entered the host, at which point it develops a defence specific to that pathogen. The cells of the adaptive immune system are called lymphocytes, the two main categories of which are B cells and T Cells. B cells are involved in the creation of neutralising antibodies that circulate in blood plasma and lymph and form part of the humoral immune response. T cells play a role in both the humoral immune response and in cell-mediated immunity. There are several subsets of activator or effector T cells, including cytotoxic T cells (CD8+) and “helper” T cells (CD4+), of which there are two main types known as Type 1 helper T cells (Th1) and Type 2 helper T cell (Th2).

Th1 cells promote a cell-mediated adaptive immune response, which involves the activation of macrophages and stimulates the release of various cytokines, such as IFNγ, TNF-α and IL-12, in response to an antigen. These cytokines influence the function of other cells in the adaptive and innate immune responses, and result in the destruction of micro-organisms. Generally, Th1 responses are more effective against intracellular pathogens, such as viruses and bacteria present inside host cells. A Th2 response, however, is characterised by the release of IL-4, which results in the activation of B cells to make neutralising antibodies, which lead the humoral immunity. Th2 responses are more effective against extracellular pathogens, such as parasites and toxins located outside host cells. Accordingly, the humoral and cell-mediated responses provide quite different mechanisms against an invading pathogen.

The present invention is concerned with the development of novel therapies for the treatment of a broad range of viral infections, including acute viral infections, and especially respiratory disorders they cause. An acute viral infection is characterized by rapid onset of disease, a relatively brief period of symptoms, and resolution normally within days. It is usually accompanied by early production of infectious virions and elimination of infection by the host immune system. Acute viral infections are typically observed with pathogens such as influenza virus and rhinovirus. Acute viral infections can be severe, a notable example being the H1N1 influenza virus, which caused the 1918 Spanish flu pandemic.

Acute infections begin with an incubation period, during which the viral genomes replicate and the host innate responses are initiated. The cytokines produced early in infection lead to classical symptoms of an acute infection: aches, pains, fever, and nausea. Some incubation periods are as short as 1 day (influenza, rhinovirus), indicating that the symptoms are produced by local viral multiplication near the site of entry. An example of a classic acute infection is uncomplicated influenza. Virus particles are inhaled in droplets produced by sneezing or coughing, and begin replicating in ciliated columnar epithelial cells of the respiratory tract. As new infectious virions are produced, they spread to neighboring cells. Virus can be isolated from throat swabs or nasal secretions from day 1 to day 7 after infection. Within 48 hours after infection symptoms appear, and these last about 3 days and then subside. The infection is usually cleared by the innate and adaptive responses in about 7 days. However, the patient usually feels unwell for several weeks, a consequence of the damage to the respiratory epithelium by the cytokines produced during infection.

Acute viral infections, such as influenza and measles, are responsible for epidemics of disease involving millions of individuals each year. When vaccines are not available, acute infections are difficult to control. This makes it exceedingly difficult to control acute infections in large populations and crowded areas. The frequent outbreak of norovirus gastroenteritis, a classic acute infection, highlights the problem. Antiviral therapy cannot be used, because it must be given early in infection to be effective. There is thus little hope of treating most acute viral infections with antiviral drugs until rapid diagnostic tests become available. However, it should be noted that there are currently no antivirals for most common acute viral diseases. There is, therefore, clearly a need in the art for improved medicaments for use in the treatment of viral infections, and especially acute viral infections.

The inventors have determined that certain related 1-phenyl-2-amino ethane derivatives have the properties required to be useful in treating such infections.

Thus, according to a first aspect of the invention, there is provided a compound of formula I:

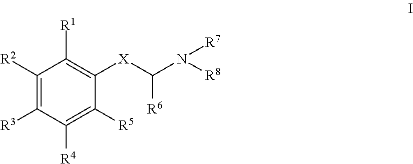

wherein:
X is CO, CHOH or CH₂;
R¹ is H, or combined with R²;
R² is H, OH, a halogen, a substituted or unsubstituted amino group, a C₁₋₅ alkyl or alkoxyl group, optionally substituted with one or more O, OH, amino and/or optionally C₁₋₃ alkyl substituted phenyl group, or combined with R¹;
R³ and R⁴ are each independently H, OH, a halogen, a substituted or unsubstituted amino group, or a C₁₋₅ alkyl or alkoxyl group, optionally substituted with one or more O, OH, amino and/or optionally C₁₋₃ alkyl substituted phenyl group;
R⁵ is H;
R⁶ is H, a C₁₋₅ alkyl group, or combined with R⁸;
R⁷ is H, or combined with R⁸;
R⁸ is combined with R⁶ or R⁷, or is a straight chain, branched or cyclo-C₁-C₉ alkyl group, optionally including one or more hetero atom in its carbon skeleton and optionally substituted with one or more OH, and/or C₅₋₆ aryl group, optionally substituted with one or more OH or C₁₋₅ alkoxyl or alkyl group;
when combined, R¹ and R², together with the associated ring carbon atoms, form an optionally O substituted cycloalkyl, cycloalkenyl, cycloheteroalkyl or cycloheteroalkenyl group of 5 or 6 carbon atoms, or 4 or 5 carbon atoms and a hetero atom;
when combined, R⁶ and R⁸, together with the nitrogen atom carrying R⁸ and the carbon atom carrying R⁶, form a 5 or 6 membered cycloheteroalkyl group; and,
when combined, R⁷ and R⁸, together with the nitrogen atom carrying them, form an optionally benzyl substituted 5 or 6 membered cycloheteroalkyl group;
or a pharmaceutically acceptable salt or solvate thereof;
for use in treating an acute viral infection.

In a second aspect of the invention, there is provided a method of preventing, treating and/or ameliorating an acute viral infection, the method comprising administering, to a subject in need of such treatment, a therapeutically effective amount of a compound as previously defined.

R² can be a hydroxyalkyl group, or include a carbonyloxy group and is, preferably, H, OH, HOCH₂—, O═CHNH—, CH₃PhCOO—, NH₂COO—, or a halogen, preferably chlorine. R² is more preferably H, OH or Cl. R₃ is preferably H, NH₂, OH or CH₃PhCOO—. R³ is more preferably H, NH₂ or OH. R⁴ is preferably H, OH, NH₂COO—, or a halogen, preferably, chlorine. R⁴ is more preferably H or Cl. R⁶ is preferably methyl, ethyl, or H, more preferably, methyl or ethyl and most preferably methyl. R⁷ is preferably H. R⁸ is preferably straight chain or branched C₂-C₆ alkyl group, optionally substituted with OH, phenyl, PhOH or PhOCH₃. R⁸ is more preferably tert. butyl, isopropyl, —C(CH₃)₂OH, —CH₂PhOCH₃, —(CH₂)₂PhOH, —CH(CH₃)CH₂CH₂Ph, or —CH(CH₃)CH₂CH₂PhOH and, most preferably, tert. butyl, —C(CH₃)₂OH, —(CH₂)₂PhOH, —CH(CH₃)CH₂CH₂Ph, or —CH(CH₃)CH₂CH₂PhOH. R⁸ can also be:

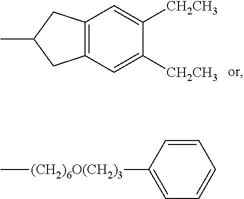

When combined, R¹ and R² preferably form the group:

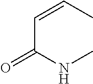

When R⁶ and R⁸ are combined it is preferred that, together with the nitrogen atom carrying R⁸ and the carbon atom carrying R⁶, they form a cycloheteroalkyl group of 5 carbon atoms and 1 nitrogen atom. When R⁷ and R⁸ are combined it is preferred that they form the group:

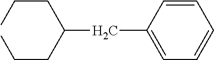

In the foregoing, Ph means phenyl and it is preferred that, when bi-substituted, any such phenyl group is 1,4-substituted.

In preferred embodiments, the present invention involves a compound of formula I wherein:
X is CO, CHOH or CH₂;
R¹ is H;
R² is H, OH, or a halogen;
R³ is H, OH or NH₂;
R⁴ is H, or a halogen;
R⁵ is H;
R⁶ is H, methyl or ethyl, or combined with R⁸;
R⁷ is H, or combined with R⁸;
R⁸ is combined with R⁶ or R⁷, or is tert. butyl, —C(CH₃)₂OH, —(CH₂)₂PhOH, —CH(CH₃)CH₂CH₂Ph, or —CH(CH₃)CH₂CH₂PhOH;
when combined, R⁶ and R⁸, together with the nitrogen atom carrying R⁸ and the carbon atom carrying R⁶, form a cycloheteroalkyl group of 5 carbon atoms and 1 nitrogen atom; and,
when R⁷ and R⁸ are combined they form the group:

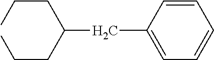

or a pharmaceutically acceptable salt or solvate thereof.

In all embodiments of the invention where R⁶ is not combined with R⁸, it is preferred for R⁶ to be a methyl or an ethyl group, preferably a methyl group. In such preferred embodiments, it is also preferred that R¹, R⁴, R⁵ and R⁷ are H, R² is H or OH, and R³ is OH. In such preferred embodiments R⁸ can be —(CH₂)₂PhOH or —CH(CH₃)CH₂CH₂PhOH.

It is known that, during an acute viral infection, such as influenza, the virus is predominantly fought through the host's innate immune system and the cell-mediated, Th1 response, and subsequently by the humoral, antibody-driven Th2 response. Furthermore, the inventors believe that, in susceptible individuals (i.e. the young, and fit and healthy individuals), the Th1 response to an influenza infection can be extremely strong, and can give rise to a so-called “cytokine storm”, involving a significant increase in the concentration of certain cytokines, such as IFN-γ and TNF-α. This “cytokine storm” can result in serious inflammation of infected lung tissue, the leakage of fluid into the lungs and significant damage to the lungs of an infected individual. The end result can be a respiratory disorder, such as pulmonary oedema or a secondary bacterial infection, which can eventually kill the infected individual, rather than the virus itself.

Baumgarth and Kelso (J. Virol., 1996, 70, 4411-4418) reported that neutralisation of the Th1 cytokine, IFN-γ, can lead to a significant reduction in the magnitude of the cellular infiltrate in lung tissue following infection, and suggested that IFN-γ may be involved in the mechanisms that regulate increased leukocyte traffic in the inflamed lung. They also postulated that IFN-γ affects the local cellular response in the respiratory tract, as well as the systemic humoral response to influenza virus infection. Based on the findings of this study, the inventors of the present invention considered whether suppression of the cytokines, IFN-γ and TNF-α, may be useful for treating influenza.

As described in the Examples, the inventors studied the effects of two related 1-phenyl-2-amino ethane derivatives (i.e. dobutamine and ritodrine), on blood cells that had been stimulated in such a way that they reflected an acute viral infection. As a model of viral infection, they used blood cell samples that had been stimulated with mitogens (lipopolysaccharide or Concanavalin A), compounds that trigger signal transduction pathways, and which thereby stimulate lymphocytes present in the blood sample to commence mitosis. This model therefore closely replicates the processes that are induced by a viral infection, and enables the direct assessment of the immune response exhibited by the lymphocytes upon treatment with the test compounds, dobutamine and ritodrine.

As described in Examples 1 to 3, the inventors found, using this in vitro model, that the 1-phenyl-2-amino ethane derivatives they tested effectively and potently inhibited the production of the cytokines, IFN-γ and TNF-α. Thus, the invention is based on the control of the Th1 immune system, which is driven by IFN-γ, and which is responsible for the hyperimmune cell-mediated response that causes respiratory collapse in susceptible individuals (e.g. the young and healthy).

These compounds are representative of a family of active compounds that share a common 1-phenyl-2-amino ethanol, ethanal or ethane core structure and which are known to exhibit similar physiological activities. This family of compounds is defined by formula (I) and it follows, because they all share the same activity providing motif, that they can all be effectively used to prevent IFN-γ and TNF-α levels from rising in the “cytokine storm” following a viral infection.

Furthermore, as described in Example 4, the inventors have also demonstrated, in an in vivo mouse model, that these compounds may be used to prevent, treat or ameliorate respiratory diseases caused by viral infections.

The inventors therefore believe that they are the first to demonstrate that, in addition to sharing other properties, the defined 1-phenyl-2-amino ethanol, ethanal and ethane derivatives can be used to modulate TNF-α and IFN-γ in such a way so as to be useful in the treatment of acute and chronic viral infections. In particular, these compounds may be used to combat respiratory disorders that are caused by acute viral infections, and which, in some cases (e.g. influenza infections), can cause death.

Various metabolites of compound (I) (i.e. any compound of formula (I)) may also be used for treating viral infections. Compound (I), for use, in the invention, may be chiral. Hence, the compound (I) may include any diastereomer and enantiomer of the formula represented by (I). Diastereomers or enantiomers of (I) are believed to display potent cytokine modulatory activity, and such activities may be determined by use of appropriate in vitro and in vivo assays, which will be known to the skilled technician. It will also be appreciated that compounds for use in the invention may also include pharmaceutically active salts, e.g. the hydrochloride.

Ritodrine and dobutamine are both 1-phenyl-2-amino ethane derivatives, and share this common structural motif with many β-adrenergic receptor agonists (also known as β-agonists). Hence, in embodiments of the invention, compound (I) may be a β-adrenergic receptor agonist. The agonist may be a β1- or β2-agonist. Examples of suitable known β2-adrenergic receptor agonists, which may be used in accordance with the invention, include salbutamol, levosalbutamol, terbutaline, pirbuterol, procaterol, metaproterenol (or orciprenaline), fenoterol, bitolterol mesylate, salmeterol, formoterol, bambuterol, clenbuterol, indacaterol, isoprenaline, rimiterol, ifenprodil, buphenine, dobutamine, and ritodrine.

In another embodiment, the compound represented by formula (I) may be the drug that is known and available under the trade name bupropion. Bupropion is known to be metabolised in vivo into a number of different metabolites also of formula (I). Therefore, buproprion or any of these metabolites may also be used for treating acute viral infections in accordance with the invention. Bupropion is metabolised non-stereoselectively to a number of enantiomers, but these compounds represent a relatively small proportion of the total metabolism of the parent drug. Compounds defined by formula (I) can therefore include these metabolites as racemates or as pairs of diastereoisomers or individual enantiomers, including the threo- and erythro-pair of diastereoisomers and the individual threo and erythro enantiomers. It is preferred that the compound defined by formula (I) includes the erythro enantiomer or enantiomers.

Exemplary bupropion metabolites include 2-(1,1-dimethylethyl)amino-1-(3-chlorophenyl)propan-1-ol, 2-(1,1-dimethyl-2-hydroxyethyl)amino-1-(3-chlorophenyl)propan-1-one, (1S,2R)-erythro-2-(1,1-dimethylethyl)amino-1-(3-chlorophenyl)propan-1-ol, (1R,2S)-erythro-2-(1,1-dimethylethyl)amino-1-(3-chlorophenyl)propan-1-ol, (1S,2S)-threo-2-(1,1-dimethylethyl)amino-1-(3-chlorophenyl)propan-1-ol and (1R,2R)-threo-2-(1,1-dimethylethyl)amino-1-(3-chlorophenyl)propan-1-ol.

In another embodiment, compound (I) may be hydrobupropion (i.e. 2-(1,1-dimethylethyl)amino-1-(3-chlorophenyl)propan-1-ol). One isomer of hydrobupropion may be (+)-threo-hydrobupropion, i.e. (R,R-hydrobupropion), and another isomer may be erythro-hydrobupropion, i.e. (R,S-hydrobupropion).

Bupropion has been previously indicated as being potentially useful for treating HSV1 and HSV2 infections, and certain bupropion metabolites only have been suggested as being potentially useful for treating inflammatory disorders. Thus, collectively in the prior art, buproprion and its metabolites, 2-(1,1-dimethylethyl)amino-1-(3-chlorophenyl)propan-1-ol, (1S,2R)-erythro-2-(1,1-dimethylethyl)amino-1-(3-chlorophenyl)propan-1-ol and (1R,2S)-erythro-2-(1,1-dimethylethyl)amino-1-(3-chlorophenyl)propan-1-ol, have previously been indicated as being potentially useful for the treatment of chronic viral infections, i.e. HSV1 and HSV2 infections.

Therefore, in a third aspect of the invention, there is provided a compound represented by the general formula I as previously defined, for use in the treatment of a viral infection, with the proviso that the compound is not bupropion or 2-(1,1-dimethylethyl)amino-1-(3-chlorophenyl)propan-1-ol in any form.

Furthermore, in a fourth aspect of the invention, there is provided a method of preventing, treating and/or ameliorating a viral infection, the method comprising administering, to a subject in need of such treatment, a therapeutically effective amount of a compound represented by the general formula I as previously defined, with the proviso that the compound is not bupropion or 2-(1,1-dimethylethyl)amino-1-(3-chlorophenyl)propan-1-ol in any form.

It is preferred that the bupropion metabolite employed in any aspect of the invention is an R enantiomer, either at the first and/or second position.

The inventors believe that the compound of formula (I) may be used in the treatment of any number of acute or chronic viral infections, and respiratory disorders which may result therefrom. The compound (I) may be used as a prophylactic (to prevent the development of a viral infection) or may be used to treat existing viral infections. The virus may be any virus, and may be an enveloped virus. The virus may be an RNA virus or a retrovirus.

For example, the viral infection, which may be treated, may be a paramyxovirus or an orthomyxovirus infection. The virus causing the infection may be a poxvirus, iridovirus, thogavirus, or torovirus. The virus causing the infection may be a filovirus, arenavirus, bunyavirus, or a rhabdovirus. It is envisaged that the virus may be a hepadnavirus, coronavirus, or a flavivirus. The invention extends to the treatment of infections with derivatives of any of the viruses disclosed herein. The term “derivative of a virus” can refer to a strain of virus that has mutated from an existing viral strain.

The virus may be selected from the group of viral genera consisting of Influenzavirus A; Influenzavirus B; Influenzavirus C; Isavirus and Thogotovirus, or any derivative of the foregoing viruses. Influenza viruses A-C include viruses that cause influenza in vertebrates, including birds (i.e. avian influenza), humans, and other mammals. Influenzavirus A causes all flu pandemics and infect humans, other mammals and birds. Influenzavirus B infects humans and seals, and Influenzavirus C infects humans and pigs. Isaviruses infect salmon, and thogotoviruses infect vertebrates (including human) and invertebrates.

Thus, compound (I) may be used to treat an infection of any of Influenzavirus A, Influenzavirus B, or Influenzavirus C, or a derivative thereof. It is preferred that compound (I) may be used for treating an infection of Influenza A, or a derivative thereof. Influenza A viruses are classified, based on the viral surface proteins hemagglutinin (HA or H) and neuraminidase (NA or N). Sixteen H subtypes (or serotypes) and nine N subtypes of influenza A virus have been identified. Thus, compound (I) may be used to treat an infection of any serotype of Influenzavirus A selected from the group of serotypes consisting of: H1N1; H1N2; H2N2; H3N1; H3N2; H3N8; H5N1; H5N2; H5N3; H5N8; H5N9; H7N1; H7N2; H7N3; H7N4; H7N7; H9N2; and H10N7, or a derivative thereof. The inventors believe that compound (I) may be particularly useful for treating viral infections of H1N1 virus, or a derivative thereof. It will be appreciated that swine flu is a strain of the H1N1 virus.

The inventors have found that, following infection with a virus, IFN-γ and TNF-α can cause fluid to leak into the lungs of an infected subject, which results in respiratory disorders that can cause eventual death. Although they do not wish to be bound by hypothesis, the inventors believe that compound (I) may be used to treat viral infections because it can act as an inhibitor of cytokine production, and in particular IFN-γ and TNF-α, and that, therefore, it can be used to treat the respiratory disorder caused by a viral infection.

The compound (I) may therefore be used to ameliorate inflammatory symptoms of virally-induced cytokine production. The anti-inflammatory compound may have an effect on any cytokine. However, preferably it modulates IFN-γ and/or TNF-α. The compound (I) may be used to treat inflammation in an acute viral infection of a naïve subject. The term “naïve subject” can refer to an individual who has not previously been infected with the virus. It will be appreciated that once an individual has been infected with a virus, such as herpes, that individual will always retain the infection.

It is especially intended that the compound (I) may be used to treat the final stages of a viral infection, such as the end stages of influenza. The compound represented by formula I may also be used to treat a viral flare-up. A “viral flare-up” can refer to either the recurrence of disease symptoms, or an onset of more severe symptoms.

The prior art does not disclose the use of any buproprion metabolite, such as 2-(1,1-dimethylethyl)amino-1-(3-chlorophenyl)propan-1-ol, or 2-(1,1-dimethyl-2-hydroxyethyl)amino-1-(3-chlorophenyl)propan-1-one, for treating any viruses of the herpes family, such as HSV 1 or HSV 2.

Thus, in a further aspect, there is provided 2-(1,1-dimethylethyl)amino-1-(3-chlorophenyl)propan-1-ol, or 2-(1,1-dimethyl-2-hydroxyethyl)amino-1-(3-chlorophenyl)propan-1-one, for use in the treatment of a viral infection caused by a herpes virus.

Also, in another aspect, there is provided a method of preventing, treating and/or ameliorating a viral infection caused by a herpes virus, the method comprising administering, to a subject in need of such treatment, a therapeutically effective amount of a 2-(1,1-dimethylethyl)amino-1-(3-chlorophenyl)propan-1-ol or 2-(1,1-dimethyl-2-hydroxyethyl)amino-1-(3-chlorophenyl)propan-1-one.

The viral infection may be caused by a herpes virus selected from the group consisting of Herpes zoster, Herpes Simplex Virus type 1 (HSV1), Herpes Simplex Virus type 2 (HSV2), Herpes labialis, human and murine cytomegalovirus, Varicella zoster virus, Epstein barr virus and human herpes virus, types 6 and 8. The herpes virus may be a herpes simplex virus, and may be HSV1 or HSV2.

(1S,2R)-erythro-2-(1,1-dimethylethyl)amino-1-(3-chlorophenyl)propan-1-ol, (1R,2S)-erythro-2-(1,1-dimethylethyl)amino-1-(3-chlorophenyl)propan-1-ol, (1S,2S)-threo-2-(1,1-dimethylethyl)amino-1-(3-chlorophenyl)propan-1-ol or (1R,2R)-threo-2-(1,1-dimethylethyl)amino-1-(3-chlorophenyl)propan-1-ol may be used to treat the viral infection caused by a herpes virus.

However, it is preferred that (1S,2R)-erythro-2-(1,1-dimethylethyl)amino-1-(3-chlorophenyl)propan-1-ol, or (1R,25)-erythro-2-(1,1-dimethylethyl)amino-1-(3-chlorophenyl)propan-1-ol is used to treat the viral infection caused by a herpes virus.

It will be appreciated that the compound of formula (I) may be used to treat viral infections in a monotherapy (i.e. use of the compound (I) alone). Alternatively, the compound (I) may be used as an adjunct to, or in combination with, known therapies used in antiviral therapy (e.g. acyclovir, gangcylovir, ribavirin, interferon, nucleotide or non-nucleoside inhibitors of reverse transcriptase, protease inhibitors and fusion inhibitors).

The compound of formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle for medicaments according to the invention should be one which is well tolerated by the subject to whom it is given, and preferably enables delivery of the agents across the blood-brain barrier, or directly to the site infected by the virus, such as the lungs.

Compositions comprising the compound of formula (I) may be used in a number of ways. For instance, oral administration may be required in which case the compound may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). Alternatively, the composition comprising (I) may be administered by inhalation (e.g. intranasally, or by mouth).

Compositions may also be formulated for topical use. For instance, ointments may be applied to the skin, areas in and around the mouth or genitals to treat specific viral infections. Topical application to the skin is particularly useful for treating viral infections of the skin or as a means of transdermal delivery to other tissues.

It will be appreciated that the amount of compound (I) that is required is determined by its biological activity and bioavailability, which in turn depends on the mode of administration, the physicochemical properties of the compound and whether the compound is being used as a monotherapy, or in a combined therapy. The frequency of administration will also be influenced by the above-mentioned factors and particularly the half-life of compound (I) within the subject being treated.

Optimal dosages to be administered may be determined by those skilled in the art, and will vary with the particular compound (I) in use, the strength of the preparation, the mode of administration, and the advancement of the disease condition. Additional factors depending on the particular subject being treated will result in a need to adjust dosages, including subject age, weight, gender, diet, and time of administration.

It will be appreciated that a skilled person will be able to calculate required doses, and optimal concentrations of compound (I) at a target tissue, based upon the pharmacokinetics of the peptides. Known procedures, such as those conventionally employed by the pharmaceutical industry (eg in vivo experimentation, clinical trials, etc.), may be used to establish specific formulations of compound (I) and precise therapeutic regimes (such as daily doses of the compounds and the frequency of administration).

Generally, a daily dose of between 0.001 μg/kg of body weight and 20 mg/kg of body weight of the compound (I) may be used for the prevention and/or treatment of a viral infection depending upon which compound is used. Suitably, the daily dose is between 0.01 μg/kg of body weight and 10 mg/kg of body weight, more suitably between 0.01 μg/kg of body weight and 1 mg/kg of body weight or between 0.1 μg/kg and 100 μg/kg body weight, and most suitably between approximately 0.1 μg/kg and 10 μg/kg body weight.

Daily doses of compound (I) may be given as a single administration (e.g. a single daily injection or a single inhalation). A suitable daily dose may be between 0.07 μg and 700 mg (i.e. assuming a body weight of 70 kg), or between 0.70 μg and 500 mg, or between 10 mg and 450 mg. The medicament may be administered before or after infection with the virus. The medicament may be administered within 2, 4, 6, 8, 10 or 12 hours after infection. The medicament may be administered within 14, 16, 18, 20, 22, or 24 hours after infection. The medicament may be administered within 1, 2, 3, 4, 5, or 6 days after infection, or at any time period therebetween.

Independently of whether or not the influenza is a pandemic influenza, the subject is someone treated with medicaments comprising compound (I) in whom symptoms of respiratory difficulty arise and/or in whom cytokine levels (any of the above mentioned cytokines, but typically IFN-α, or TNF-γ) increase at the onset of symptoms of respiratory difficulty. More preferably, the subject is a subject in whom symptoms of respiratory difficulty arise, and/or in whom cytokine levels increase, at the following times after onset of influenza symptoms: from 12, 24, 18 or 36 hours or more (more preferably from 48 hours or more, from 60 hours or more, or from 72 hours or more; most preferably from 36-96 hours, from 48-96 hours, from 60-96 hours or from 72-96 hours). Alternatively, and independently of whether or not the influenza is a pandemic influenza, the subject is someone in whom symptoms of respiratory difficulty arise and/or in whom cytokine levels increase, at the onset (or early stage) of recruitment of the adaptive immune system into the infected lung.

As described in the in vivo mouse studies of Example 4, the inventors have shown that mice that were administered more than one dose of a cytokine inhibitor showed improvement to symptoms of the influenza infection. Therefore, it is envisaged that medicaments comprising compound (I) may be administered more than once to the subject in need of treatment. The compound may require administration twice or more times during a day. As an example, compound (I) may be administered as two (or more depending upon the severity of the viral infection being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter, and so on. It is envisaged that the compound may be administered every day (more than once if necessary) following viral infection.

Thus, the compound (I) is preferably suitable for administration to a subject as described above, preferably suitable for administration at the aforementioned points after the onset of influenza symptoms.

Alternatively, a slow release device may be used to provide optimal doses of compounds according to the invention to a patient without the need to administer repeated doses.

Based on their findings that the compounds described herein may be used to reduce the levels of cytokines, such as TNF-α and IFN-γ, the inventors believe that these effects of the compounds may be harnessed and used in the manufacture of clinically useful compositions.

Hence, in a fifth aspect there is provided a pharmaceutical composition comprising a therapeutically effective amount of a compound represented by the general formula I, as previously defined, and a pharmaceutically acceptable vehicle, for use in the treatment of viral infections.

The infection may be acute or chronic.

A “therapeutically effective amount” of a compound represented by formula (I) is any amount which, when administered to a subject, results in decreased levels of cytokines, such as TNF-α and IFN-γ, and thereby provides prevention and/or treatment of an acute viral infection.

For example, the therapeutically effective amount of compound (I) used may be from about 0.07 μg to about 700 mg, and preferably from about 0.7 μg to about 70 mg. The amount of compound (I) is from about 7 μg to about 7 mg, or from about 7 μg to about 700 μg.

A “subject” may be a vertebrate, mammal, or domestic animal, and is preferably a human being. Hence, medicaments according to the invention may be used to treat any mammal, for example human, livestock, pets, or may be used in other veterinary applications.

A “pharmaceutically acceptable vehicle” as referred to herein is any combination of known compounds known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agent (i.e. the compound (I) according to the invention). In tablets, the active agent may be mixed with a vehicle having the necessary compression properties in suitable proportions and compacted in the shape and size desired. The powders and tablets preferably contain up to 99% of the active agent. Suitable solid vehicles include, for example calcium phosphate, magnesium stearate, talc, sugars, lactose, dextrin, starch, gelatin, cellulose, polyvinylpyrrolidine, low melting waxes and ion exchange resins.

In another embodiment, the pharmaceutical vehicle may be a gel and the composition may be in the form of a cream or the like. In yet another embodiment, the pharmaceutical vehicle may be a liquid, and the pharmaceutical composition may be in the form of a solution. Liquid vehicles are used in preparing solutions, suspensions, emulsions, syrups, elixirs and pressurized compositions. The active compound (I) may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intravenous and particularly subcutaneous injection. The compound (I) according to the invention may be prepared as a sterile solid composition that may be dissolved or suspended at the time of administration using sterile water, saline, or other appropriate sterile injectable medium.

The compound (I) may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compound (I) can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Embodiments of the invention will now be further described, by way of example only, with reference to the following Examples, and to the accompanying diagrammatic drawings, in which:

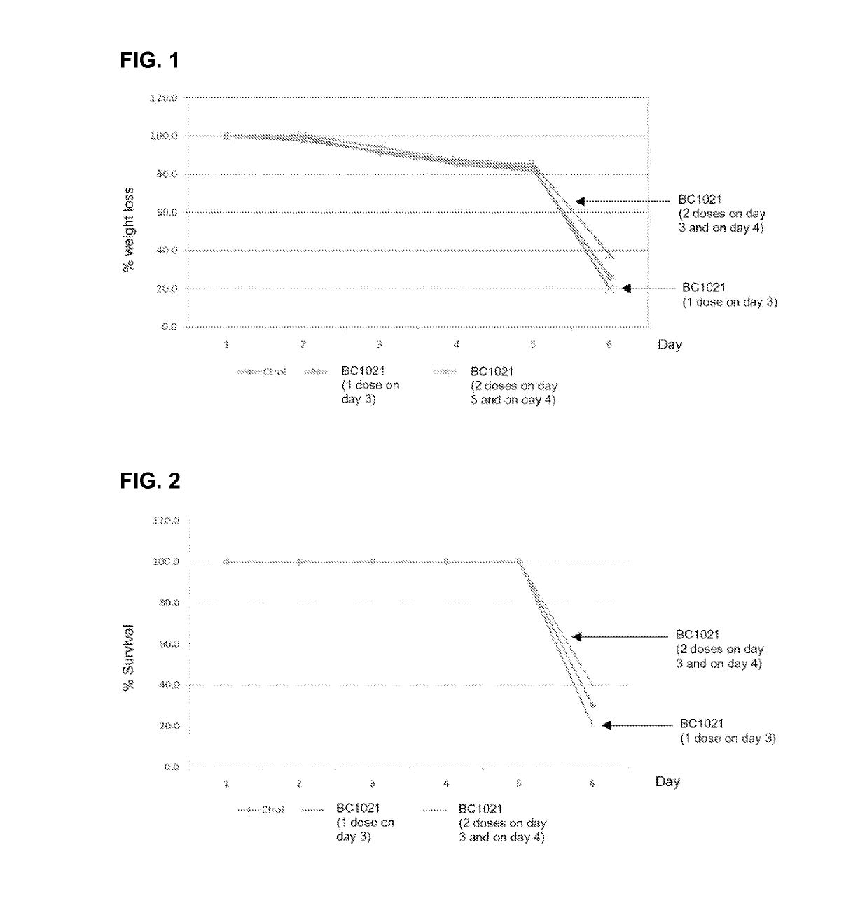

FIG. 1 is graph showing the results of an in vivo mouse challenge, in which mice were infected with a H1N1 virus, and then treated with a compound represented by formula I, i.e. dobutamine (BC1021). Dobutamine was administered to the mice as a single dose on day 3, and as a double dose, on days 3 and 4, and the weight loss of the mice was measured. No dobutamine was added to the control mice;

FIG. 2 is a graph showing the survival rate of mice in the in vivo mouse challenge described in relation to FIG. 1. The mice were administered with dobutamine as a single dose on day 3, and on days 3 and 4, and the percentage rate of survival was measured. No dobutamine was added to the mice of the control;

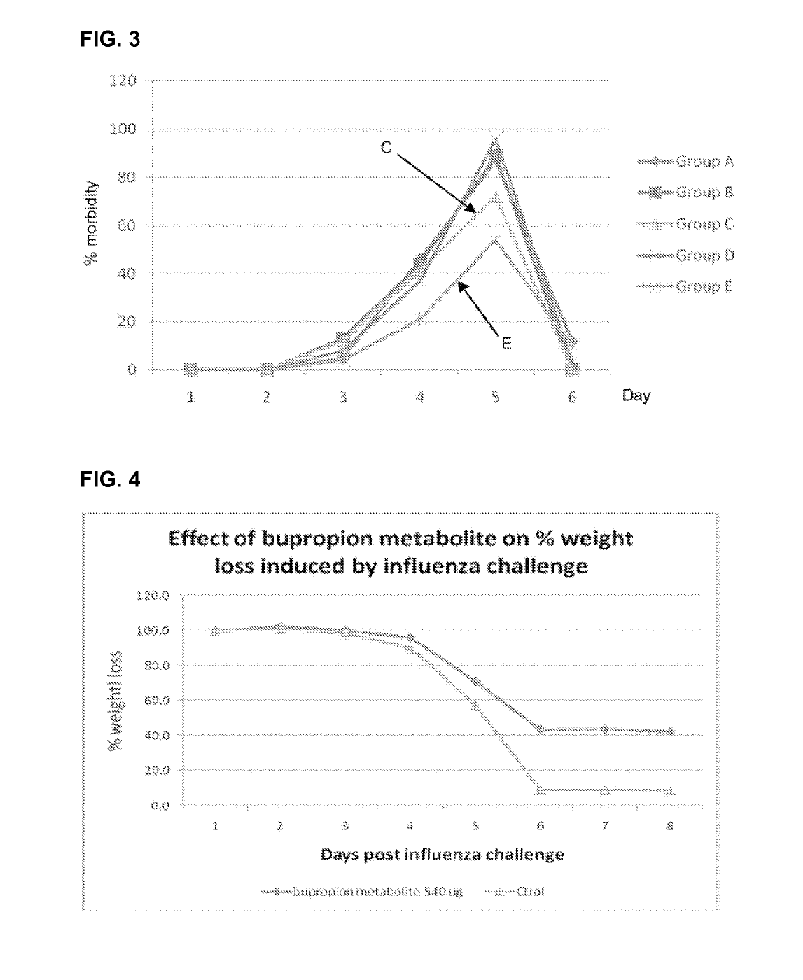

FIG. 3 is a graph showing Sum Total Morbidity (not mortality) of the in vivo mouse challenge described in relation to FIG. 1. The effects on morbidity (i.e. a general measure of the well-being of the mouse) of single doses (on day 3) and double doses (on days 3 and 4) of compounds represented by formula I, i.e. dobutamine (BC1021) and ritodrine (BC1023), were measured. Line A: Control (no drug added); Line B: BC1021, 1 dose on day 3; Line C: BC1021, 2 doses, on days 3 and 4; Line D: BC1023, 1 dose on day 3; Line E: BC1023, 2 doses, on days 3 and 4;

FIG. 4 is graph showing the results of an in vivo mouse challenge, in which mice were infected with a H1N1 virus, and then treated with a compound represented by formula I, i.e. 2-(1,1-dimethylethyl)amino-1-(3-chlorophenyl)propan-1-ol, which is one embodiment of a bupropion metabolite (BC1053). The bupropion metabolite was administered to the mice as a single dose on day 3, and as a double dose, on days 3 and 4, and the weight loss of the mice was measured. No metabolite was added to the control mice;

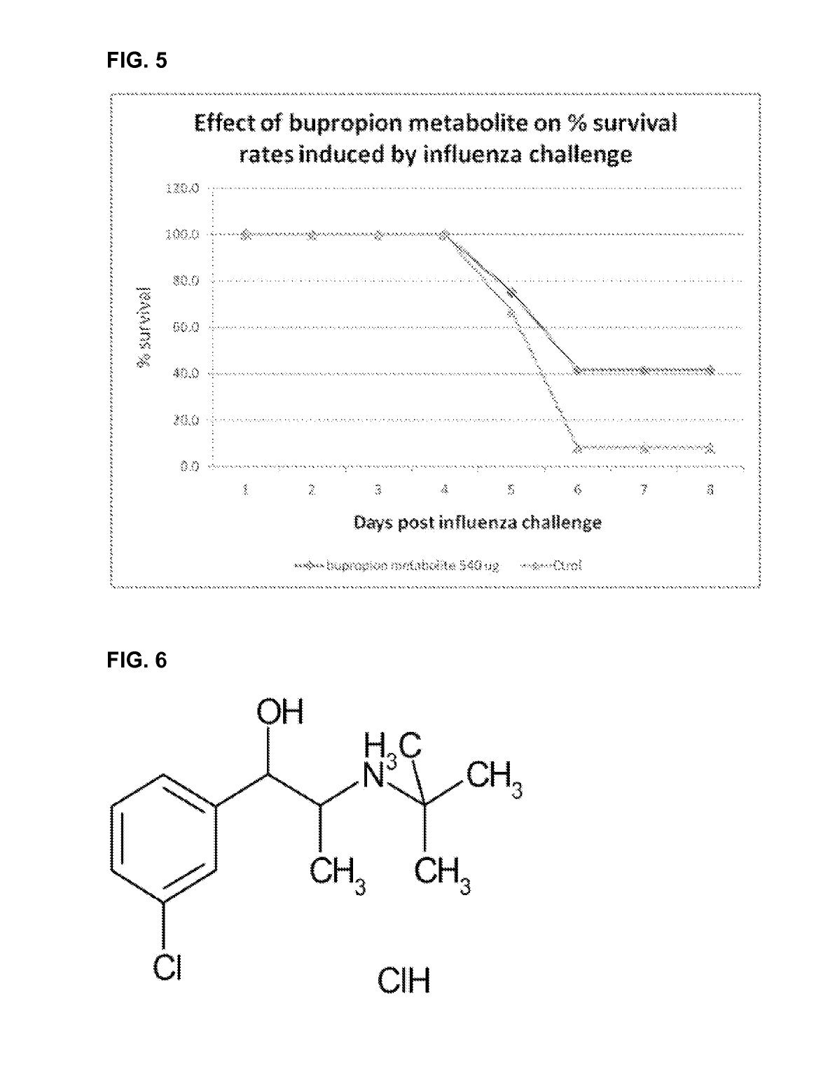

FIG. 5 is a graph showing the survival rate of mice in the in vivo mouse challenge described in relation to FIG. 4. The mice were administered with the bupropion metabolite as a single dose on day 3, and on days 3 and 4, and the percentage rate of survival was measured. No metabolite was added to the mice of the control; and

FIG. 6 shows the chemical structure of one embodiment of another embodiment of a compound represented by formula I (e.g. a bupropion metabolite, denoted herein as BC1053).

### EXAMPLES

The inventors carried out a range of in vitro and in vivo experiments in order to determine the effects of various compounds represented by formula I on the production of the cytokines, IFN-γ and TNF-α. The inventors have demonstrated in the results described below that both ritodrine, dobutamine and a bupropion metabolite, (2-(1,1-dimethylethyl)amino-1-(3-chlorophenyl)propan-1-ol, denoted herein as BC1053) surprisingly act as inhibitors of IFN-γ and TNF-α. Furthermore, they have demonstrated in in vivo mouse models that administration of ritodrine, dobutamine and the bupropion metabolite results in a reduction in the viral symptoms (i.e. reduction in weight loss, increase in survival rate, and reduction in total morbidity) in mice.

Materials and Methods

1) Isolation, Culture and Treatment of Peripheral Blood Mononuclear Cells (PBMC)

Blood was collected in 6 ml vacutainers (green cap). Blood was processed within 2 h of collection. Materials used: Non-coagulated blood; FCS; RPMI-1640 media supplemented with L-Gln and P/S; PBS; sterile tips and pipettes; Sterile 15 ml Falcon; Sterile V-bottom 96-well plates with lids; Neubauer chamber; Trypan Blue solution; 70% IPA solution; Accuspin-Histopaque tubes (Sigma, A7054)

Procedure:
1. Dilute samples 1:1 in sterile PBS;
2. Add 30 ml of diluted blood into an Accuspin-Histopaque tube (Sigma, A7054);
3. Centrifuge at 800 rcf 15min at room temperature (RT);
4. After centrifugation, the red blood cells will remain at the bottom below the frit. The monocytes (PBMC) will be present on a layer above the frit, with the plasma on top;
5. Collect the PBMC layer with a pipette into a fresh 15 ml Falcon tube and top up to 15 ml of PBS;
6. Centrifuge at 250 rcf 10 min at RT;
7. Discard the supernatant, flick the pellet and add another 10 ml of PBS;
8. Centrifuge at 250 rcf 10 min at RT;
9. Repeat steps 7 and 8;
10. Discard the supernatant and resuspend the pellet in 1 ml of complete medium (RPMI 1640 10% FCS);
11. Count cells and make a 4×10⁶ cell/ml suspension in complete medium. Add 100 μl of cell suspension per well in a V-bottom 96-well plate. Then add 50 μl of stimulant or vehicle in complete media, and 50 μl of drug or vehicle in complete media. Incubate the cells for 24 h at 37° C. 5% CO₂;
12. After incubation, take 60 μl of cell supernatant to measure IFNγ and TNFα by ELISA (OptEIA human IFNγ, cat No. 555142 and human TNF, Cat No. 555212) following manufacturer's instructions (BD Biosciences);

2) Human Umbilical Vein Endothelial Cells (HUVEC) Cell Seeding Protocol

Materials: HUVEC (ECACC 200-05n); M199 medium (Sigma M2154); L-Glutamine solution 200 mM (Sigma G7513) ; Penicillin/streptomycin (Sigma, P0781); Gentamicin/amphotericin B (Invitrogen, from LSGS kit# S003K); Human epidermal growth factor (hEGF) (Invitrogen, from LSGS kit# S003K); Basic fibroblast growth factor (bHGF) (Invitrogen, from LSGS kit# S003K); Heparin. (Invitrogen, from LSGS kit# S003K); Trypsin 10× solution (Sigma T4174); Sterile PBS (Sigma D8537); EDTA 0.02% solution (Sigma E8008); Fetal Bovine Serum (Sigma F9665)

HUVEC complete growth media: M199 medium containing 10% Foetal Calf Serum, 100 U/ml penicillin/0.1 mg/ml streptomycin, 2 mM L-Glutamine, 10 μg/ml gentamicin, 0.25 μg/ml amphotericin B, 10 ng/ml human epidermal growth factor (hEGF), 3 ng/ml basic fibroblast growth factor (bHGF) and 10 μg/ml heparin.

Procedure:
1. Harvest cells by trypsinisation when they are 80% confluent.
   a. Remove medium;
   b. Wash cell monolayer with 0.02% EDTA and dispose (4 ml for T75);
   c. Pipette 5 ml 1×Trypsin solution in PBS and rock gently to ensure the solution covers all the cells;
   d. Remove 4.5 ml of the trypsin solution immediately;
   e. Re-cap the flask and monitor the trypsinisation under a microscope;
   f. When cells become rounded release them by hitting the side of the flask against your palm until the cells detach;
   g. Add FCS to the flask and mix (2 ml for T75);
   h. Harvest the cell suspension into a 15 ml Falcon and top up the volume with 10 ml serum free medium;
   i. Centrifuge at 250 g 5 min;
   j. Dispose of the supernatant, flick the pellet and resuspend it in 1 ml of complete medium.
   k. Count the cells using the haemocytometer and prepare the required volume of cell suspension at 2.5×10⁴ cells/ml.
2. Seed flat bottom 96-well plates with 200 ul of cell suspension per well (=5×10³ cells/well). Incubate plates for 4-5 days at 37° C. and 5% CO₂, until >80% confluency is reached, changing the media every other day where possible.

3) HUVEC Stimulation/Treatments Protocol

Materials: M199 medium (Sigma M2154); L-Glutamine solution 200 mM (Sigma G7513); Penicillin/streptomycin (Sigma, P0781); Gentamicin/ amphotericin B (Invitrogen, from LSGS kit# S003K); Human epidermal growth factor (hEGF) (Invitrogen, from LSGS kit# S003K); Basic fibroblast growth factor (bHGF) (Invitrogen, from LSGS kit# S003K); Heparin (Invitrogen, from LSGS kit# S003K); Sterile PBS (Sigma D8537); Fetal Bovine Serum (Sigma F9665); Ibuprofen (Sigma I110); Ethanol (Fisher E/0600/17); DMSO (Sigma D4540); TNF-α, human, natural (NIBSC 88/786); Pipettes and sterile pipette tips; Sterile universals or Falcon tubes; Sterile 1.5 ml screwcap tubes; 70% Isopropanol solution; Virkon.

HUVEC complete growth media: M199 medium containing 10% Foetal Calf Serum, 100 U/ml penicillin/0.1 mg/ml streptomycin, 2 mM L-Glutamine, 10 μg/ml gentamicin, 0.25 μg/ml amphotericin B, 10 ng/ml human epidermal growth factor (hEGF), 3 ng/ml basic fibroblast growth factor (bHGF) and 10 μg/ml heparin.

Stimulant/treatments: TNF-α (100 U/ml); Test compounds (100, 10, & 1 μM) with TNF-α (100 U/ml); Test compounds (100, 10, & 1 μM) only; Compound vehicle controls (0.5% DMSO and 0.1% ethanol); Ibuprofen control (1 mM) with TNF-α (100 U/ml); Ibuprofen control (1 mM) only; Complete media only

Procedure:
1. Prepare TNF-α at 100 U/ml in complete HUVEC growth media;
2. Prepare fresh stock of test compounds at 20 mM in DMSO;
3. Prepare test concentrations of the compounds (100, 10 and 1 μM) in complete HUVEC growth media with and without TNF-α;
4. Prepare ibuprofen control stock at 1M in ethanol. Dilute to 1 mM in complete HUVEC growth media with and without TNF-α;
5. Prepare the controls for the drug vehicles (0.5% DMSO and 0.1% ethanol) in complete HUVEC growth media;
6. Remove media from wells and add 150 μl of the tests and controls, in triplicates.
7. Incubate plate/s for 18 hours at 37° C. and 5% CO₂.

4) Vascular Cell Adhesion Molecule-1 (V-CAM-1) ELISA Protocol

Kit used: DuoSet human VCAM-1 Elisa Set (R&D Systems DY809)

Buffers:

Wash buffer—PBS/0.05% Tween 20 (Sigma P9416)

Assay diluent—PBS/1% BSA (Sigma A30590

Substrate solution—TMB solution (Sigma T0440)

Stop solution—2N H₂SO₄

Capture Antibody—stock @360 μg/ml. Dilute to working concentration of 2 μg/ml (1:180)

Detection Antibody—stock @36 μg/ml. Dilute to working concentration of 200 ng/ml (1:180)

Standards—stock @70 ng/ml. Top standard @1000 μg/ml (1:70)

Streptavidin-HRP—Dilute 1:200

Procedure:
1. Dilute capture antibody in PBS;
2. Coat plate with 100 μl/well;
3. Seal plate and incubate overnight @room temperature;
4. Empty wells and wash 3× with wash buffer. Blot dry;
5. Add 180 μl/well of assay diluent, to block the plate. Seal the plate and incubate for 1 hr @room temperature;
6. Empty wells and wash 3× with wash buffer. Blot dry;
7. Add standards (duplicates), samples (triplicates) and controls (triplicates) 60 μl/well. Seal the plate and incubate for 2 hr @room temperature;
8. Empty wells and wash 3× with wash buffer. Blot dry;
9. Add detection antibody 60 μl/well. Seal the plate and incubate for 2 hr @room temperature;
10. Empty wells and wash 5× with wash buffer, leaving plate to soak for at least 30 seconds between each wash. Blot dry;
11. Add streptavidin-HRP 60 μl/well. Seal the plate and incubate for 20 min @room temperature;
12. Empty the wells and wash 5× with wash buffer, leaving plate to soak for at least 30 seconds between each wash. Blot dry;
13. Add TMB substrate solution 60 μl/well. Seal the plate and incubate for 20 min @room temperature;
14. Add 30 μl/well of stop solution;
15. Read absorbance @450 nm.

5) In Vivo Mouse Studies Using Dobutamine

Protocol: Fifty (50) C57BL/6 female mice (6-7 weeks old), were divided into four experimental groups containing ten (10) animals each. On day 1, animals received an intranasal lethal dose (50 μl total, 25 μl nostril) of Influenza A/PR/8/34 under halothane induced anesthesia. On Day 3, animals received one intra-peritoneal injection (100-150 μl) of the test compound. On Day 4 or 5, all animals still alive received a second intra-peritoneal injection (100-150 μl) of the test compound.

All animals were assessed daily for morbidity, weight loss and survival from Day 1 until at least Day 6. Morbidity variables (i.e. Body Condition, Posture, Activity, Piloerection, Respiration, Vocalisation, Ataxia and Oculo/Nasal Discharges) were recorded according to the following scale of severity: Normal (0), Mild (1), Laboured (2) and Severe/Cull-point (3).

6) In Vivo Mouse Studies Using Bupropion Metabolite

The bupropion metabolite known as 2-(1,1-dimethylethyl)amino-1-(3-chlorophenyl)propan-1-ol (BC1053), in 10% ethanol, was delivered orally to influenza infected mice, as follows.

Two groups of n=12 C57BLK/6 barrier reared female mice (6-8 weeks old, and between 15-18 g in weight) were intranasally challenged with a lethal dose of influenza (A/PR/8/34). On day 3 post-challenge, the animals received the following treatments: group A received an oral gavage of 100 μl of 10% Ethanol in water; and group B received an oral gavage of 100 μl containing 540 μg of bupropion metabolite in 10% Ethanol in water.

The animals were weighed and monitored daily for morbidity and mortality up to day 6, when all animals were culled. Average weight loss per group and survival were calculated.

### Example 1

### Stimulation Experiments Using Mitogens, LPS and Con A

Plasma B cells can enter mitosis when they encounter an antigen matching their immunoglobulin. A mitogen is a chemical substance that triggers signal transduction pathways in which mitogen-activated protein kinase is involved, thereby encouraging a cell to commence cell division, leading to mitosis. Thus, mitogens can be effectively used to stimulate lymphocytes and therefore assess immune function. By stimulating lymphocytes, mitogens can be used to replicate the effects of a viral infection.

The two mitogens that the inventors used to stimulate lymphocytes, and therefore assess immune function, were lipopolysaccharide (LPS) and Concanavalin A (Con A). LPS acts on B cells but not T cells, whereas Con A acts on T cells but not B cells. The effects of two embodiments of the compound represented by formula I, i.e. dobutamine (referred to in the tables as BC1021) and ritodrine (BC1023), on the levels of IFN-γ and TNF-α were investigated in LPS and Con A stimulated assays. Peripheral Blood Mononuclear Cells (PMBC) were independently administered with each mitogen, LPS or Con A, and then treated with either dobutamine or ritodrine. Control experiments were conducted in which no LPS or Con A was added, such that any effect on the levels of IFN-γ and TNF-α could be directly attributed to the presence of the test compound, dobutamine or ritodrine.

LPS Stimulation Studies

The results of the LPS stimulation experiments are shown in Tables 1 and 2. The values in the Tables are expressed as the percentage value of the LPS only control. Thus, the maximum concentration of the cytokine, either IFN-γ or TNF-α, expressed from the PMBC cells in the presence of only LPS is said to be 100%, and the concentrations of the cytokines that are expressed from the PMBC cells in the presence of (i) LPS and (ii) either dobutamine or ritodrine, are expressed as percentage of the LPS only 100% control. Standard deviation values (s.d.) are given underneath each value of expressed IFN-γ levels.

TABLE 1
Determination of IFN-γ levels under LPS stimulation (Percentage
IFN-γ levels compared to 100% untreated cells under LPS stimulation)

| | | BC1021 | | | BC1023 | |
|---|---|---|---|---|---|---|
| +LPS | | (dobutamine) + LPS | | | (ritodrine) + LPS | |
| | 100 μM | 10 μM | 1 μM | 100 μM | 10 μM | 1 μM |
| 100.00% | 37.22% | 29.84% | 82.88% | 38.74% | 51.41% | 44.79% |
| 42.81 | 15.51 | 6.04 | 26.61 s.d. | 15.51 s.d. | 13.69 | 9.28 |
| s.d. | s.d. | s.d. | | | s.d. | s.d. |
| | BC1021 | | | | | |
| | only | BC1023 only | | | | |
| untreated | 100 μM | 10 μM | 1 μM | 100 μM | 10 μM | 1 μM |
| 0.00% | −0.99% | −0.33% | 1.21% | 5.46% | 2.73% | 1.04% |
| 0.72 | 1.06 | 1.29 | 0.55 s.d. | 0.55 s.d. | 1.74 | 0.85 |
| s.d. | s.d. | s.d. | | | s.d. | s.d. |

With reference to the data shown in Table 1, the inventors were surprised to observe that the concentration of IFN-γ was decreased in the presence of either dobutamine or ritodrine in LPS stimulated cells.

TABLE 2
Determination of TNF-α levels under LPS stimulation (Percentage TNF-α levels
compared to 100% untreated cells under LPS stimulation)

| | | | | | BC1023 | |
|---|---|---|---|---|---|---|
| +LPS | | BC1021 (dobutamine) + LPS | | | (ritodrine) + LPS | |
| | 100 μM | 10 μM | 1 μM | 100 μM | 10 μM | 1 μM |
| 100.00% | 81.41% | 84.79% | 91.20% | 85.74% | 85.01% | 62.12% |
| 6.28 s.d. | 2.95 s.d. | 2.31 s.d. | 6.41 s.d. | 7.09 s.d. | 4.25 s.d. | 2.01 s.d. |
| | BC1021 | | | | | |
| | only | BC1023 only | | | | |
| untreated | 100 μM | 10 μM | 1 μM | 100 μM | 10 μM | 1 μM |
| 0.00% | −151.15% | −145.07% | −80.18% | −56.57% | 0.74% | −29.75% |
| 9.82 s.d. | 0.62 s.d. | 1.70 s.d. | 9.61 s.d. | 10.00 s.d. | 14.95 s.d. | 8.13 s.d. |

With reference to the data shown in Table 2, the inventors were also surprised to observe that the concentration of TNF-α was also decreased in the presence of either dobutamine or ritodrine in LPS stimulated cells. The negative values in the controls suggest that the levels of cytokine are rising. However, since the concentrations of cytokines are very low at the end of the treatment, the increase is only minimal.

Con A Stimulation Studies

The results of the Con A experiments are illustrated in Tables 3 and 4.

TABLE 3
Determination of TNF-α levels under Con A stimulation (Percentage TNF-α
levels compared to 100% untreated cells under Con A stimulation)

| | | BC1021 (dobutamine) + | | | | |
|---|---|---|---|---|---|---|
| +Con A 5 | | ConA | | | BC1023 (ritodrine) + ConA | |
| | 100 μM | 10 μM | 1 μM | 100 μM | 10 μM | 1 μM |
| 100.00% | −33.95% | 102.36% | 103.05% | 104.93% | 106.20% | 103.92% |
| 2.31 s.d. | 1.44 s.d. | 2.66 s.d. | 1.53 s.d. | 2.50 s.d. | 2.22 s.d. | 4.87 s.d. |
| | BC1021 | | | | | |
| | only | BC1023 only | | | | |
| untreated | 100 μM | 10 μM | 1 μM | 100 μM | 10 μM | 1 μM |
| 0.00% | −42.84% | −33.21% | −35.44% | −14.80% | −24.69% | −5.94% |
| 6.39 s.d. | 1.10 s.d. | 4.03 s.d. | 2.55 s.d. | 18.73 s.d. | 2.81 s.d. | 6.46 s.d. |

With reference to the data shown in Table 3, the inventors observed that the concentration of TNF-α was also decreased in the presence of either dobutamine or ritodrine in Con A stimulated cells.

TABLE 4
Determination of IFN-γ levels under Con A stimulation (Percentage IFN-γ
levels compared to 100% untreated cells under Con A stimulation)

| | | | | | BC1023 | |
|---|---|---|---|---|---|---|
| | | BC1021 (dobutamine) + | | | (ritodrine) + | |
| +Con A 5 | | ConA | | | ConA | |
| | 100 μM | 10 μM | 1 μM | 100 μM | 10 μM | 1 μM |
| 100.00% | −1.80% | 32.77% | 63.40% | 41.69% | 34.25% | 39.93% |
| 18.07 s.d. | 0.42 | 4.60 s.d. | 11.35 s.d. | 6.02 | 2.35 | 9.81 |
| | s.d. | | | s.d. | s.d. | s.d. |
| | BC1021 | | | | | |
| | only | BC1023 only | | | | |
| untreated | 100 μM | 10 μM | 1 μM | 100 μM | 10 μM | 1 μM |
| 0.00% | −3.26% | −2.51% | −2.61% | −1.87% | −1.52% | −0.86% |
| 0.29 s.d. | 0.75 | 0.46 s.d. | 0.62 s.d. | 0.21 | 0.35 | 1.09 |
| | s.d. | | | s.d. | s.d. | s.d. |

With reference to the data shown in Table 4, the inventors were very surprised to observe that the concentration of IFN-γ was decreased in the presence of either dobutamine or ritodrine in Con A stimulated cells. In particular, the inventors observed that the dose of 100 μM dobutamine had a significant effect in decreasing the concentration of IFN-γ.

### Example 2

### Determination of the Percentage Cell Survival of Con A Stimulated Cells

The inventors measured the cell survival rate of Con A stimulated cells, and the results are shown in Table 5.

TABLE 5
Percentage cell survival compared to 100% untreated cells under Con A
stimulation

| | BC1021 (dobutamine) + | | | BC1023 | | |
|---|---|---|---|---|---|---|
| | Con A (5 μg/ml) | | | (ritodrine) + Con A (5 μg/ml) | | |
| +Con A 5 | 100 μM | 10 μM | 1 μM | 100 μM | 10 μM | 1 μM |
| 98.33% | 48.22% | 71.40% | 81.09% | 75.27% | 79.76% | 80.61% |
| 1.68 s.d. | 2.32 | 1.52 s.d. | 2.78 | 2.72 | 2.77 | 5.80 |
| | s.d. | | s.d. | s.d. | s.d. | s.d. |
| | BC1021 | | | | | |
| | only | BC1023 only | | | | |
| Untreated | 100 μM | 10 μM | 1 μM | 100 μM | 10 μM | 1 μM |
| 98.81% | 44.95% | 69.80% | 76.89% | 72.56% | 75.25% | 78.69% |
| 1.06 s.d. | 2.78 | 1.86 s.d. | 1.04 | 2.57 | 2.18 | 2.59 |
| | s.d. | | s.d. | s.d. | s.d. | s.d. |

As can be seen in Table 5, for all doses of ritodrine and dobutamine, percentage cell survival was higher in the presence of either ritodrine or dobutamine compared to the untreated controls. Accordingly, the inventors have demonstrated that administration of either compound to Con A stimulated cells results in a higher survival rate than untreated controls.

### Example 3

### Determination of Cell Cytotoxicity When Exposed to Digitonin Under Con A Stimulation

Digitonin is a glycoside obtained from Digitalis purpurea, which acts as a detergent, and effectively water-solubilizes lipids in the plasma membrane. Therefore, digitonin can be used to permeabilise cell membranes. The inventors therefore investigated digitonin's cell membrane-permeabilising effects on Con A-stimulated cells to determine the cytotoxic effects of ritodrine or dobutamine. Table 6 shows the results.

TABLE 6
Percentage cytotoxicity compared to 100% for digitonin under Con A
stimulation

| | | BC1021 | | BC1023 | | | |
|---|---|---|---|---|---|---|---|
| | | (dobutamine) + | | (ritodrine) + | | | |
| +Con A 5 | | Con A (5 μg/ml) | | Con A (5 μg/ml) | | untreated | |
| | 100 μM | 10 μM | 1 μM | 100 μM | 10 μM | 1 μM | |
| 50.35% | 38.65% | 32.21% | 34.99% | 34.44% | 35.55% | 33.39% | 49.86% |
| 1.46 s.d. | 1.66 s.d. | 1.14 s.d. | 1.68 s.d. | 1.35 s.d. | 1.55 s.d. | 2.36 s.d. | 3.05 s.d. |

| | BC1021 | | | | | |
|---|---|---|---|---|---|---|
| | only | BC1023 only | | | | |
| untreated | 100 μM | 10 μM | 1 μM | 100 μM | 10 μM | 1 μM |
| 52.09% | 32.33% | 42.94% | 47.71% | 43.81% | 43.63% | 44.54% |
| 1.78 s.d. | 1.14 s.d. | 0.85 s.d. | 1.05 s.d. | 1.79 s.d. | 1.70 s.d. | 1.68 s.d. |

With reference to Table 6, in the vast majority of cases, cytoxicity rates were lower in samples treated with either ritodrine or dobutamine. The only exception was for the 100 μM dose of dobutamine, but the inventors believe that this result is statistically insignificant.

### Example 4

### In Vivo Mouse Studies Using Dobutamine

Using standard techniques as described above, mice were infected with a H1N1 virus which was allowed to become established in each of the subjects. Each test mouse was then treated with dobutamine (BC1021) either with a single dose on day 3 after infection with the virus, or as two doses, one on day 3 and one on day 4 after infection. In the control mice, no dobutamine was administered. The weight loss of both treated and untreated mice was then determined.

As shown in FIG. 1, the mice that received two doses of dobutamine (on days 3 and 4 after infection with the virus) showed at least a 10% lower reduction in weight loss than the control mice. Accordingly, although the inventors do not wish to be bound by hypothesis, they believe that the reduced levels of the cytokines, IFN-γ and TNF-α, in H1N1-infected mice upon exposure to dobutamine results in the mice maintaining their weight. The inventors believe that the single dose of dobutamine had little effect on the mice because it has a short half-life.

Referring to FIG. 2, there are shown the results of percentage survival of mice treated with dobutamine. As can be seen in FIG. 2, mice treated with two doses of dobutamine, one on day 3 and one on day 4, showed a higher survival rate than the control, untreated mice. Again, the inventors postulate that the short half-life of dobutamine was to blame for the single dose of this compound having little effect on the mice.

The inventors also investigated the effects of dobutamine (single and double doses) as well as ritodrine (single and double doses) on the Sum Total Morbidity of the tested mice. Referring to FIG. 3, there are shown the data of these experiments. The value of Sum Total Morbidity corresponds to a confidence value of the general “wellness” of the mice, and takes into account the quality of the fur and grooming of the mice, and whether or not the mice are able to feed and walk. Measurement of Morbidity values will be known to the skilled technician. As can be seen in FIG. 3, all doses of both dobutamine and ritodrine, whether single or double, resulted in an improvement of the morbidity value of the treated mice, giving a clear indication that the viral symptoms had been reduced. However, it is particularly noteworthy that mice that had been given two doses of dobutamine and ritodrine (i.e. Groups C and E), showed the most improvements, with Group E (i.e. doses with dobutamine on days 3 and 4) being the most effective.

### Example 5

### In Vivo Mouse Studies Using Bupropion Metabolite

As described above, mice were infected with a H1N1 virus which was allowed to become established in each of the subjects. Each test mouse was then treated with 2-(1,1-dimethylethyl)amino-1-(3-chlorophenyl)propan-1-ol (i.e. a bupropion metabolite, BC1053) with a single dose on day 3 after infection with the virus. In the control mice, no metabolite was administered. The weight loss of both treated and untreated mice was then determined.

As shown in FIG. 4, the mice that received a dose of the bupropion metabolite (on day 3 after infection with the virus) showed at least a 30% lower reduction in weight loss than the control mice. Accordingly, although the inventors do not wish to be bound by hypothesis, they believe that the reduced levels of the cytokines, IFN-γ and TNF-α, in H1N1-infected mice upon exposure to the bupropion metabolite results in the mice maintaining their weight.

Referring to FIG. 5, there are shown the results of percentage survival of mice treated with the bupropion metabolite. As can be seen in FIG. 5, mice treated with the metabolite showed a much higher (about 30%) survival rate than the control, untreated mice.

### SUMMARY

In summary, the inventors were surprised to observe that both dobutamine and ritodrine acted as cytokine inhibitors (i.e. IFN-γ and TNF-α), especially given the poor pharmacokinetics of these two drugs. They therefore believe that any compound represented by formula (I) may be used as an IFN-γ and TNF-α inhibitor, which can be used in the treatment of a viral infection, such as influenza. The encouraging results of the in vivo mouse studies described in Example 4 clearly demonstrate that mice infected with a H1N1 virus can be effectively treated by administration of either single, but especially double, doses of dobutamine or ritodrine. Hence, it is clear that any compound (I) could be used to treat viral infections.

1-34. (canceled)

35. A method of treating an acute viral infection in a subject in need thereof, the method comprising the step of administering to the subject a therapeutically effective amount of a compound of formula I, or a pharmaceutically acceptable salt or solvate thereof, wherein the acute viral infection is caused by a herpes virus, wherein formula I has the structure: 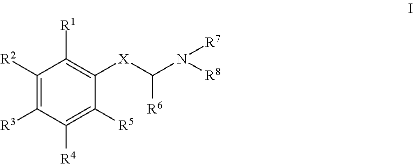
wherein:
X is CO, CHOH or CH₂;
R¹ is H, or combined with R²;
R² is H, OH, a halogen, a substituted or unsubstituted amino group, a C₁₋₅ alkyl or alkoxyl group, optionally substituted with one or more O, OH, amino and/or optionally C₁₋₃ alkyl substituted phenyl group, or combined with R¹;
R³ and R⁴ are each independently H, OH, a halogen, a substituted or unsubstituted amino group, or a C₁₋₅ alkyl or alkoxyl group, optionally substituted with one or more O, OH, amino and/or optionally C₁₋₃ alkyl substituted phenyl group;
R⁵ is H;
R⁶ is H, a C₁₋₅ alkyl group, or combined with R⁸;
R⁷ is H, or combined with R⁸;
R⁸ is combined with R⁶ or R⁷, or is a straight chain, branched or cyclo-C₁-C₉ alkyl group, optionally including one or more hetero atom in its carbon skeleton and optionally substituted with one or more OH, and/or C₅₋₆ aryl group, optionally substituted with one or more OH or C₁₋₅ alkoxyl or alkyl group;
when combined, R¹ and R², together with the associated ring carbon atoms, form an optionally O substituted cycloalkyl, cycloalkenyl, cycloheteroalkyl or cycloheteroalkenyl group of 5 or 6 carbon atoms, or 4 or 5 carbon atoms and a hetero atom;
when combined, R⁶ and R⁸, together with the nitrogen atom carrying R⁸ and the carbon atom carrying R⁶, form a 5 or 6 membered cycloheteroalkyl group; and
when combined, R⁷ and R⁸, together with the nitrogen atom carrying them, form an optionally benzyl substituted 5 or 6 membered cycloheteroalkyl group; and
wherein administration of the compound reduces a symptom associated with the acute viral infection, thereby treating the subject.

36. The method according to claim 35, wherein R² is a hydroxyalkyl group or a carbonyloxy group.

37. The method according to claim 35, wherein R² is H, OH, Cl, HOCH₂—, O═CHNH—, CH₃PhCOO—, NH₂COO—, or a halogen.

38. The method according to claim 35, wherein R₃ is H, NH₂, OH or CH₃PhCOO—.

39. The method according to claim 35, wherein R³ is H, NH₂ or OH.

40. The method according to claim 35, wherein R⁴ is H, OH, Cl, NH₂COO—, or a halogen.

41. The method according to claim 35, wherein R⁶ is methyl, ethyl, or H.

42. The method according to claim 35, wherein R⁷ is H.

43. The method according to claim 35, wherein R⁸ is a straight chain or branched C₂-C₆ alkyl group, optionally substituted with OH, phenyl, PhOH or PhOCH₃,

44. The method according to claim 35, wherein R⁸ is tert-butyl, isopropyl, —C(CH₃)₂OH, —CH₂PhOCH₃, —(CH₂)₂PhOH, —CH(CH₃)CH₂CH₂Ph, or —CH(CH₃)CH₂CH₂PhOH.

45. The method according to claim 35, wherein R⁸ is

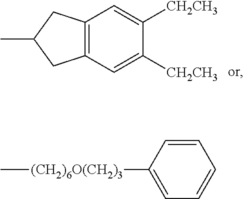

46. The method according to claim 35, wherein when combined, R¹ and R² form the group

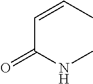

47. The method according to claim 35, wherein when R⁶ and R⁸ are combined, together with the nitrogen atom carrying R⁸ and the carbon atom carrying R⁶, they form a cycloheteroalkyl group of 5 carbon atoms and 1 nitrogen atom.

48. The method according to claim 35, wherein when R⁷ and R⁸ are combined they form the group:

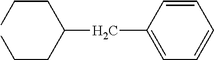

49. The method according to claim 35, wherein the compound comprises any diastereomer and enantiomer of formula I.

50. The method according to claim 35, wherein the compound is a β2-adrenergic receptor agonist.

51. The method according to claim 50, wherein the β2-adrenergic receptor agonist is albutamol, levosalbutamol, terbutaline, pirbuterol, procaterol, metaproterenol (or orciprenaline), fenoterol, bitolterol mesylate, salmeterol, formoterol, bambuterol, clenbuterol, indacaterol, isoprenaline, rimiterol, ifenprodil, buphenine, dobutamine or ritodrine.

52. The method according to claim 35, wherein the compound is bupropion or a metabolite thereof.

53. The method according to claim 52, wherein the bupropion metabolite is 2-(1,1-dimethylethyl)amino-1-(3-chlorophenyl)propan-1-ol, 2-(1,1-dimethyl-2-hydroxyethyl)amino-1-(3-chlorophenyl)propan-1-one, (1S,2R)-erythro-2-(1,1-dimethylethyl)amino-1-(3-chlorophenyl)propan-1-ol, (1R,2S)-erythro-2-(1,1-dimethylethyl)amino-1-(3-chlorophenyl)propan-1-ol, (1S,2S)-threo-2-(1,1-dimethylethyl)amino-1-(3-chlorophenyl)propan-1-ol, or (1R,2R)-threo-2-(1,1-dimethylethyl)amino-1-(3-chlorophenyl)propan-1-ol.

54. The method according to claim 35, wherein the compound is not bupropion, or 2-(1,1-dimethylethyl)amino-1-(3-chlorophenyl)propan-1-ol in any form.

55. The method according to claim 35, wherein the compound is not bupropion, or 2-(1,1-dimethylethyl)amino-1-(3-chlorophenyl)propan-1-ol in any form.

56. The method according to claim 35, wherein the compound is 2-(1,1-dimethylethyl)amino-1-(3-chlorophenyl)propan-1-ol, or 2-(1,1-dimethyl-2-hydroxyethyl)amino-1-(3-chlorophenyl)propan-1-one.

57. The method according to claim 35, wherein the compound is 2-(1,1-dimethylethyl)amino-1-(3-chlorophenyl)propan-1-ol, or 2-(1,1-dimethyl-2-hydroxyethyl)amino-1-(3-chlorophenyl)propan-1-one.

58. The method according to claim 35, wherein the compound is 2-(1,1-dimethylethyl)amino-1-(3-chlorophenyl)propan-1-ol, or 2-(1,1-dimethyl-2-hydroxyethyl)amino-1-(3-chlorophenyl)propan-1-one.

59. The method according to claim 35, wherein the compound is 2-(1,1-dimethylethyl)amino-1-(3-chlorophenyl)propan-1-ol, or 2-(1,1-dimethyl-2-hydroxyethyl)amino-1-(3-chlorophenyl)propan-1-one according to any one of claims 30-33, wherein (1S,2R)-erythro-2-(1,1-dimethylethyl)amino-1-(3-chlorophenyl)propan-1-ol, or (1R,2S)-erythro-2-(1,1-dimethylethyl)amino-1-(3-chlorophenyl)propan-1-ol.

60. The method according to claim 35, wherein the compound modulates IFN-γ and/or TNF-α.

61. The method according to claim 35, wherein the herpes virus is a paramyxovirus or an orthomyxovirus.

62. The method according to claim 35, wherein the herpes virus is a Herpes zoster virus, a Herpes Simplex Virus type 1 (HSV1), a Herpes Simplex Virus type 2 (HSV2), a Herpes labialis, a human cytomegalovirus, a murine cytomegalovirus, a Varicella zoster virus, a Epstein barr virus, a human herpes virus type 6, or a human herpes virus type 8.

63. The method according to claim 35, wherein the herpes virus is Influenzavirus A, Influenzavirus B, or Influenzavirus C, or a derivative thereof.

64. The method according to claim 63, wherein the Influenzavirus A is serotype of H1N1, H1N2, H2N2, H3N1, H3N2, H3N8, H5N1, H5N2, H5N3, H5N8, H5N9, H7N1, H7N2, H7N3, H7N4, H7N7, H9N2, H10N7, or a derivative thereof.

65. The method according to claim 64, wherein the herpes virus is a H1N1 virus or a derivative thereof.

66. The method according to claim 35, wherein the symptom comprises inflammation.

67. The method according to claim 35, wherein the symptom comprises an inflammatory symptom associated with virally-induced cytokine production.

68. The method according to claim 35, wherein the symptom comprises a symptom associated with a viral flare-up.

69. The method according to claim 35, wherein the subject is a naïve subject.

70. A method of treating an acute viral infection in a subject in need thereof, the method comprising the step of administering to the subject a therapeutically effective amount of 2-(1,1-dimethylethyl)amino-1-(3-chlorophenyl)propan-1-ol or 2-(1,1-dimethyl-2-hydroxyethyl)amino-1-(3-chlorophenyl)propan-1-one.
